(19) United States
(12) Patent Application Publication
(10) Pub. No.: US 2024/0000689 A1
(43) Pub. Date: Jan. 4, 2024

(54) COMPOSITIONS AND METHODS FOR STYLING HAIR

(71) Applicant: L'OREAL, Paris (FR)

(72) Inventors: Lisa YE-TSE, Brooklyn, NY (US); Vibha SHAH, Branchburg, NJ (US); Allison PERNER, Metuchen, NJ (US)

(21) Appl. No.: 17/855,767

(22) Filed: Jun. 30, 2022

(51) Int. Cl. A61K 8/60; A61Q 5/06; A61Q 5/04; A61K 8/81; A61K 8/73; A61K 8/25; A61K 8/92; A61K 8/34; A61K 8/891

(52) U.S. Cl. CPC A61K 8/60; A61Q 5/06; A61Q 5/04; A61K 8/8141; A61K 8/732; A61K 8/25; A61K 8/92; A61K 8/345; A61K 8/347; A61K 8/891

(57) ABSTRACT

The disclosure relates to compositions comprising (a) at least one latex type film former acrylate-based polymers, polyurethane-based polymers, derivatives thereof, or combinations thereof; (b) at least one polysaccharide gum; (c) potato starch or a derivative thereof; and (d) water. The disclosure also relates to methods of using the compositions to style hair.

### TECHNICAL FIELD

The present disclosure relates to compositions and methods for styling hair, particularly, to compositions and methods for temporarily straightening curly or wavy hair without requiring the use of heat.

### BACKGROUND

Consumers with curly or wavy hair often desire to temporarily straighten their hair for a change in appearance, and then switch back to their curly style without damage. In addition, reducing the curls of very curly hair may increase the manageability and ease of styling of such hair.

Currently, temporary straightening curly/wavy hair or frizzy hair typically involves the use of hot tools such as blow dryers and/or flat irons. The high temperature of the blow dryers and/or irons leads to a breakage of hydrogen bonds in the keratin of the hair, achieving a temporary straightening. The hydrogen bonds are formed again by the action of moisture, so that the hair reverts back to its original shape over the time because of air humidity or after washing or damping the hair.

Longer-term and/or permanent straightening is usually achieved by using chemical straightening treatments, such as relaxers, with high pH. The shape of the hair is largely determined by the disulfide bonds linking two cysteine moieties of the hair keratin. To achieve a longer-term and/or permanent shaping or styling effect, it usually involves cleavage of the disulfide bonds by the action of a sulfide- or thiol-group containing reducing agent or relaxing agent. After the hair has been brought into the desired shape, new disulfide bonds are formed by applying an oxidizing agent such as hydrogen peroxide, thus fixing the shape of the hair. The use of such agents, however, may cause damage to the hair and weaken the hair, resulting in subsequent breakage. Conventional products that provide hold, such as styling gels, although may also provide straightening effects to the hair, usually flake on the hair and result in very stiff and unnatural look hair. In addition, conventional hair straightening routines are usually time-consuming.

As such, there is a need for new and improved compositions that can be simply applied to hair and provide straightening effects to curly or wavy hair that would not damage the hair, while at the same time imparting various additional sensorial properties to the hair such as frizz control, softness, smoothness, and/or shine. The present disclosure addresses these needs and concerns and relates to new compositions and methods that can provide styling effect, particularly, a temporary straightening effect on curly/waving hair without the requirement of using heat.

It has now surprisingly been found that a combination of components described herein allows for a balance between straightening performance and consumer desired look and feel, even under high humidity condition, and is useful for providing desired temporarily straightening effect to curly or wavy hair through a simple application routine without the use of hot tools or harsh chemicals such as reducing agents or relaxers, while at the same time delivering additional advantageous sensorial benefits such as frizz control, natural look and feel, to the hair. The temporary straightening effect and other benefits such as frizz control imparted to the hair are lasting, such as may last under high humidity conditions and until the next hair wash, without any damage to the natural curls.

### SUMMARY

The present disclosure relates to compositions that provide temporary straightening effect to curly or wavy hair, while at the same time in some embodiments providing one or more additional benefits such as frizz control, softness, smoothness, and/or shine to the hair. The disclosure also relates to methods of using the compositions for styling hair, in particular, temporarily straightening curly/wavy hair, while at the same time in at least some embodiments providing additional sensorial benefits described above to the hair.

In various embodiments, the hair styling compositions according to the present disclosure comprise (a) at least one latex type film former chosen from acrylate-based polymers, polyurethane-based polymers, derivatives thereof, or combinations thereof; (b) at least one polysaccharide gum; (c) potato starch or a derivative thereof; and (d) water. Optionally, the compositions may comprise at least one silicone acrylate, at least one polyol, at least one silicone oil, and/or at least one non-ionic guar. The hair styling compositions may in at least certain embodiments be formulated in the form of a gel-cream, where the compositions exhibit textures like a gel with an opaque creamy appearance. The compositions typically have a pH of less than 7, such as, for example, from about 4 to about 6, or from about 5 to about 6. The hair styling compositions according to the present disclosure may in at least some embodiments be free or essentially free of chemical relaxing agents or reducing agents. In some embodiments, the hair styling compositions may be free or essentially free of polyphenols, including tannic acid. Additionally, the compositions disclosed herein may be free or substantially free of cationic polymers. The compositions may also be free or essentially free of dyes, pigments, and/or colorants.

In various embodiments, the total amount of the latex type film former(s) may range from about 0.5% to about 15% by weight relative to the total weight of the composition. In further embodiments, at least one of the latex type film former is chosen from acrylates copolymer of two or more monomers of (meth)acrylic acid or one of their simple ester, styrene/acrylates copolymer, acrylates/ethylhexyl acrylate copolymer, or mixtures thereof. In some embodiments, at least one latex type film former is acrylates copolymer.

The at least one polysaccharide gum, in various embodiments, may be present in an amount ranging from about 0.1% to about 2% by weight, relative to the total weight of the composition. In further embodiments, the at least one polysaccharide gum may be chosen from sclerotium gum, gum arabic, tragacanth gum, karaya gum, guar gum, gellan gum, tara gum, locust bean gum, tamarind gum, xanthan gum, carrageenan gum dehydroxanthan gum, seneca gum, gellan gum, or mixtures thereof. In certain embodiments, the polysaccharide gum comprises, consists essentially of, or consists of dehydroxanthan gum.

In further embodiments, potato starch or a derivative thereof may be present in an amount ranging from about 0.1% to about 8%, for example from about 0.2% to about 5%, from about 0.3% to about 2%, or from about 0.4% to about 1% by weight, relative to the total weight of the hair styling composition. In various embodiments, water is present in an amount of at least 70% by weight, such as from about 70% to about 96%, from about 80% to about 95%, from about 85% to about 95% by weight, relative to the total weight of the composition.

If present, the at least one silicone acrylate may be in an amount ranging from about 0.5% to about 20% by weight, relative to the total weight of the hair styling composition. If present, the at least one silicone oil may be in an amount ranging from about 2% to about 10% by weight, relative to the total weight of the composition. In further embodiments, the at least one silicone oil may be chosen from methicone, amodimethicones, dimethicones, polydiethylsiloxanes, dimethiconols, phenyl trimethicone, or mixtures thereof.

If present, the total amount of polyol(s) may range from about 1% to about 10% by weight, relative to the total weight of the composition. In some embodiments, the at least one polyol is chosen from sorbitol, glycols, or mixtures thereof. The amount of sorbitol, if present, may range from about 1% to about 3% by weight, relative to the total weight of the composition. The amount of glycol(s), if present, may range from about 1% to about 5% by weight, relative to the total weight of the composition.

If present, the total amount of non-ionic guar(s) may range from about 0.1% to about 1% by weight, relative to the total weight of the composition.

In further embodiments, compositions according to the disclosure may comprise (a) at least one latex type film former chosen from acrylate-based polymers, polyurethane, derivatives thereof, or combinations thereof, present in an amount ranging from about 1% to about 15% by weight; (b) at least one polysaccharide gum, present in an amount ranging from about 0.1% to about 2% by weight; (c) potato starch or a derivative thereof, present in an amount ranging from about 0.2% to about 3% by weight; (d) optionally, a silicone acrylate, present in an amount ranging from about 1% to about 1.5% by weight; (e) optionally, at least one polyol, present in an amount ranging from about 1% to about 10% by weight; (f) optionally, at least one non-ionic guar, present in an amount ranging from about 0.1% to about 1% by weight; (g) optionally, at least one at least one silicone oil, present in an amount ranging from about 2% to about 10% by weight, relative to the total weight of the composition; and (h) water, present in an amount of at least 70% by weight; wherein the pH of the hair styling composition is less than 7; and wherein all amounts are relative to the total weight of the composition.

In various embodiments, methods according to the present disclosure comprise applying to the hair any hair styling composition disclosed herein, and after the hair is dried, straightening the hair by combing through with fingers or a coming tool. The composition applied on the hair may be left on the hair for a desired period of time until the hair is washed. In further embodiments, no heat or chemical relaxers or reducing agents are applied to the hair before, during, or after the hair styling composition is applied to the hair. As such, in at least certain embodiments, the methods do not include the use of a reducing agent or chemical relaxer, and/or a step of heating the hair.

The compositions are not mascara compositions, and are not intended to be applied to the eyelashes or eyebrows. Similarly, the compositions are not make-up compositions, and are not intended to be applied to the skin.

### BRIEF DESCRIPTION OF THE FIGURES

The accompanying figures, which are incorporated herein and constitute part of this specification, illustrate exemplary embodiments of the disclosure, and, together with the general description given above and the description provided herein, serve to demonstrate features of the disclosure.

FIG. 1 is a set of photographs showing the visual appearance of hair swatches treated with exemplary composition 1A according to the present disclosure and comparative compositions C1, C2, and C3, and a control hair swatch treated with water, while the hair is still wet (T1), after the hair was dried and detangled (T2), and after the hair was exposed to high humidity (80% relative humidity (RH)) at room temperature for 1 hour (T3).

FIG. 2 is a graph showing the graded straightness of hair swatches treated with compositions 1A and 1B according to the present disclosure in comparison with the hair swatches treated with comparative compositions C1, C2, and C3 and control hair swatches treated with water, after the hair was dried and exposed to high humidity (80% RH) at room temperature for 1 hour (T3).

FIG. 3 is a graph showing the graded frizziness of hair swatches treated with compositions 1A and 1B according to the present disclosure in comparison with the hair swatches treated with comparative compositions C1, C2, and C3 and control hair swatches treated with water, after the hair was dried and exposed to high humidity (80% RH) at room temperature for 1 hour (T3).

FIG. 4 is a set of photographs showing the side view of a consumer's hair before the hair was treated (TO) and after the hair was treated with composition 2A and composition 2B according to the present disclosure via half head application and dried (T1).

It is to be understood that the foregoing and following descriptions are exemplary and explanatory only, and are not intended to be restrictive of any subject matter claimed.

### DETAILED DESCRIPTION

The disclosure relates to compositions for styling hair, for example compositions for temporarily straightening wavy or curly hair, while imparting frizz control and other sensorial benefits to the hair. The disclosure also relates to methods of using the compositions for styling hair.

### I. Compositions

In various embodiments, compositions according to the disclosure comprise (a) at least one latex type film former chosen from acrylate-based polymers, polyurethane-based polymers, derivatives thereof, or combinations thereof; (b) at least one polysaccharide gum; (c) potato starch or a derivative thereof; and (d) water. Optionally, the compositions may comprise a silicone acrylate, at least one polyol, at least one silicone oil, and/or at least one non-ionic guar. The compositions may, for example, be formulated in the form of an oil-in-water emulsion, and may have a pH of less than 7. A polyphenol and a chemical relaxing agent or reducing agent are not required; as such, the compositions may be free or essentially free of polyphenols, chemical relaxing agents, and/or reducing agents. The compositions can, for example, be used as leave-in compositions for styling hair, particularly, for temporarily straightening curly or wavy hair, without the use of heat.

It has been surprisingly found that the compositions may provide beneficial effects to hair, such as temporarily straightening the hair, without the need for heat or chemical relaxers, and at the same time in at least some embodiments providing one or more of softness, smoothness, shine, volume control, and/or frizz control to the hair. It is believed, without being limited, that the benefits result from a synergistic combination of the ingredients disclosed herein. For example, without being limited by theory, it is believed that the acrylates copolymers form a flexible film providing styleability and the polysaccharide(s) form a strong fixative film that provides hold with improved humidity resistance properties; it is also believed that starches such as potato starch help deliver the film formers onto the hair with glide for even distribution and provides desired fiber alignment. The optional ingredients are believed, without being limited by theory, to improve the sensoriality by increasing the suppleness of the treated hair (lower rigidity) and to provide easy application and better detangling. As a result, the combination of ingredients disclosed herein allows for a balance between straightening performance and cosmeticity even under high humidity conditions.

### Latex Type Film Former

Compositions according to the disclosure comprise at least one latex type film former chosen acrylate-based polymers, polyurethane-based polymers, derivatives thereof, or combinations thereof. As used herein, polyurethane-based polymers and/or derivatives thereof are polymers contain carbamate linkages.

In some embodiments, acrylate-based polymers or derivatives used as latex type film formers may be chosen from acrylates copolymer, styrene/acrylates copolymer, acrylates/ethylhexyl acrylate copolymer, or mixtures thereof. In some instances, at least one latex type film former is acrylates copolymer, i.e., copolymers of two or more monomers consisting of acrylic acid, methacrylic acid or one of their simple esters.

In certain embodiments, the at least one latex type film former is chosen from polymers resulting from copolymerization of monomers chosen from (meth)acrylics, (meth)acrylates, and/or (meth)acrylamides. The term “(meth)acryl” and variations thereof, as used herein, means acryl or methacryl. In certain embodiments, the (meth)acrylic monomers may be chosen from acrylic acid, methacrylic acid, citraconic acid, itaconic acid, maleic acid, fumaric acid, crotonic acid, maleic anhydride, or mixtures thereof.

In certain embodiments, the (meth)acrylic monomers may be chosen from C1-C8 alkyl (meth)acrylic, methyl (meth)acrylic, ethyl (meth)acrylic, propyl (meth)acrylic, isopropyl (meth)acrylic, butyl (meth)acrylic, tert-butyl (meth)acrylic, pentyl(meth) acrylic, isopentyl (meth)acrylic, neopentyl (meth)acrylic, hexyl (meth)acrylic, isohexyl (meth)acrylic, 2-ethylhexyl (meth)acrylic, cyclohexyl (meth)acrylic, isohexyl (meth)acrylic, heptyl (meth)acrylic, isoheptyl (meth)acrylic, octyl (meth)acrylic, isooctyl (meth)acrylic, or mixtures thereof.

In certain embodiments, suitable acrylate-based polymers may be chosen from aqueous dispersions of Methacrylic Acid/Ethyl Acrylate copolymer (INCI name: Acrylates Copolymer, such as LUVIFLEX Soft sold by the company BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI name: Polyacrylate-2 Crosspolymer, such as FIXATE Superhold sold by the company Lubrizol), Butyl acrylate, PEG-10 acrylate, PPG-6 acrylate and dimethylacrylamide copolymer (INCI name: Polyacrylate-3 crosspolymer), Styrene/Acrylic copolymer (such as NEOCRYL A-1120 sold by the company DSM), Ethylhexyl Acrylate/Methyl Methacrylate/Butyl Acrylate/Acrylic Acid/Methacrylic Acid copolymer (INCI name: Acrylates/Ethylhexyl Acrylate Copolymer, such as DAITOSOL 5000SJ sold by the company Daito Kasei Kogyo), Acrylic/Acrylates Copolymer (INCI name: Acrylates Copolymer, such as DAITOSOL 5000AD sold by the company Daito Kasei Kogyo), or Acrylic copolymers and Acrylates Copolymers (such as VINYSOL 2140 sold by the company Daido Chemical, ACULYN 33 sold by the company Dow Chemical, LUVIMER MAE sold by the company BASF, or BALANCE CR sold by the company Akzo Nobel).

In various embodiments, the amount of latex type film former(s) chosen from acrylate-based polymers, polyurethane-based polymers, derivatives thereof, or combinations thereof may range from about 0.5% to about 15% by weight, including all subranges therebetween, such as, for example, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 2% to about 15%, from about 2% to about 12%, from about 2% to about 10%, from about 2% to about 9%, from about 2% to about 8%, from about 2% to about 7%, from about 2% to about 6%, from about 4% to about 12%, from about 4% to about 10%, from about 4% to about 8%, from about 5% to about 15%, from about 5% to about 12%, from about 5% to about 10%, from about 5% to about 8% by weight, relative to the total weight of the composition. In some embodiments, composition disclosed herein comprise acrylates copolymer in an amount ranging from about 0.5% to about 10%, such as from about 1% to about 10%, from about 4% to about 10%, or from about 5% to about 8% by weight, such as about 5%, about 7%, or about 9% by weight, relative to the total weight of the composition.

### Polysaccharide Gums

Compositions according to the disclosure comprise at least one polysaccharide gum. In various embodiments, suitable polysaccharide gums may be anionic gums and/or non-ionic.

Non-limiting examples of polysaccharide gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, dehydroxanthan gum, carrageenan gum, and biosacharide gum. Modified gums or derivatives of gums may also be used, such as, for example, deacylated gellan gum, welan gum, or hydroxypropylated guar gum, such as Jaguar HP 105 sold by Rhodia. In various embodiments, the polysaccharide gum(s) comprise, consist essentially of, or consist of dehydroxanthan gum.

In some embodiments, suitable polysaccharide gums that can be used are chosen from gums, such as gum arabic, tragacanth gum, karaya gum, guar gum, gellan gum, tara gum, locust bean gum, tamarind gum, xanthan gum, dehydroxanthan gum, carrageenan gum, Seneca gum, sclerotium gum, gellan gum, etc.

In various exemplary embodiments, the total amount of polysaccharide gum(s) may vary, or combinations thereof, typically ranges from about 0.1% to about 2% by weight, including all subranges therebetween, such as from about 0.2% to about 2%, from about 0.2% to about 1.5%, from about 0.2% to about 1.2%, from about 0.2% to about 1.0%, from about 0.2% to about 0.8%, from about 0.5% to about 2%, from about 0.5% to about 1.5%, from about 0.5% to about 1.2%, from about 0.5% to about 1%, from about 0.5% to about 0.8%, from about 1% to about 2%, or from about 1% to about 1.5% by weight, such as about 0.5%, about 0.75%, about 1%, or about 1.2% relative to the total weight of the composition, including all ranges and subranges thereof.

### Potato Starch or Derivatives Thereof

Compositions according to the disclosure comprise potato starch or a derivative thereof. As used herein, potato starch includes all polysaccharides formed from glucose monomers and originating in various edible tuberous plants from genus Solanum such as Solanum tuberosum.

In some embodiments, a potato starch that is used may be partially or completely modified. Modified starches are starch products that are obtained by physical, enzymatic or chemical processes which satisfy more stringent technical requirements. The grain structure and other essential properties are retained after modification. The basic material for the production of modified potato starch may be natural or degraded starch, which may be converted into the respective derivatives by polymer-analog reactions. Physically modified starches are equated with the native starches because they are only treated thermally, that is so say they are boiled.

In various exemplary embodiments, the total amount of potato starch or derivative thereof may vary, but typically ranges from about 0.2% to about 5% by weight, including all subranges therebetween, such as, for example, from about 0.2% to about 4%, from about 0.2% to about 3%, from about 0.2% to about 2%, from about 0.2% to about 1.5%, from about 0.2% to about 1.2%, from about 0.2% to about 1%, from about 0.5% to about 4%, from about 0.5% to about 3%, from about 0.5% to about 2%, from about 0.5% to about 1%, by weight, such as about 0.5%, about 0.55%, about 0.6%, about 0.65%, about 0.7%, about 0.75%, about 0.8%, about 0.85%, about 0.9%, about 0.95%, or about 1% relative to the total weight of the composition.

### Water

Compositions according to the disclosure comprise water. In various embodiments, the total amount of water is at least 70% by weight, such as from about 70% to about 96%, from about 80% to about 95%, from about 85% to about 95% by weight, relative to the total weight of the composition.

### Silicone Acrylate Copolymers

Compositions according to the disclosure may optionally comprise at least one silicone acrylate copolymer. In various embodiments, if present, the at least one silicone acrylate copolymer may be chosen from, for example, acrylates/polytrimethylsiloxymethacrylate copolymer.

For example, silicone acrylate sold under the name DOWSIL™ FA 4002 ID Silicone Acrylate may be used, which is a blend of approximately 40% of acrylates/polytrimethylsiloxy-methacrylate copolymer in isododecane.

In various embodiments, if present, the at least one silicone acrylate copolymer may present in an amount ranging from about 0.5% to about 20% by weight, including all subranges therebetween, such as, for example, from about 0.5% to about 15%, from about 0.5% to about 10%, from about 0.5% to about 8%, from about 0.5% to about 5%, from about 0.5% to about 2%, from about 0.5% to about 1.5%, from about 0.5% to about 1%, from about 0.5% to about 20% by weight, including all subranges therebetween, such as, for example, from about 0.5% to about 15%, from about 0.5% to about 10%, from about 0.5% to about 8%, from about 0.5% to about 5%, from about 0.5% to about 2%, from about 0.5% to about 1.5%, from about 0.5% to about 1%, from about 1% to about 20%, from about 1% to about 15%, from about 1% to about 10%, from about 1% to about 8%, from about 1% to about 5%, or from about 1% to about 1.5%, for example, 0.8%, 1%, 1.3%, 1.5%, 2%, 5%, 10%, 15% by weight, relative to the total weight of the hair styling composition.

### Silicone Oils

Compositions according to the disclosure may optionally comprise at least one silicone oil. In various embodiments, if present, the at least one silicone oil may be chosen from, for example, hydroxylmethylpolysiloxane (methicone), amino functional silicones such as amodimethicones, dimethicone copolyols, polydimethylsiloxanes (dimethicones), polydiethylsiloxanes, polydimethyl siloxanes having terminal hydroxyl groups (dimethiconols), phenyl trimethicone, or mixtures thereof. In some embodiments, compositions according to the disclosure may optionally comprise at least two silicone oil.

In some embodiments, the compositions include at least one high molecular weight silicone oil and at least one low molecular weight silicone oil. Such a blend of high and low molecular weight silicone oil may provide smoothing benefits along with improved humidity resistance to the hair. In some embodiments, when both high and low molecular weight silicon oils are included in the composition, these two types of silicone oils are present in amounts that are the same or similar, i.e., with a weight ratio thereof at or near 1:1.

In some embodiments, the at least one silicone oil is chosen from dimethicone and/or amodimethicone. In a further embodiment, compositions disclosed herein may comprise both dimethicone and amodimethicone, with a preferred weight ratio of dimethicone to amodimethicone at or near about 1:1.

In some embodiments, mixtures of silicone compounds, for example silicone emulsions, may be used. For example, silicone emulsions sold under the name XIAMETER™ by Dow, which are blends of Amodimethicone (and) Trideceth-12 (and) Cetrimonium Chloride or Amodimethicone (and) Trideceth-6 (and) Cetrimonium Chloride may be chosen.

In various embodiments, if present, the amount of silicone oil(s) may range from about 2% to about 10% by weight, including all subranges therebetween, such as, for example, from about 2% to about 9%, from about 2% to about 8%, from about 2% to about 7%, from about 2% to about 6%, from about 2% to about 5%, from about 4% to about 10%, from about 4% to about 9%, from about 5% to about 10%, from about 5% to about 9%, from about 5% to about 8% by weight, relative to the total weight of the composition.

### Polyols

Compositions according to the disclosure optionally comprise one or more polyols. In certain embodiments, the compositions disclosed herein may comprise at least two polyols. As used herein, the term “polyol” refers to an organic molecule comprising at least two free hydroxyl groups. The polyols may be liquid at ambient temperature (25° C.). Suitable polyols that may be comprised in the compositions disclosed herein may be glycols or compounds with numerous hydroxyl groups. In some cases, the at least on polyol is chosen from polyols having 2-32 carbon atoms, i.e., C2-C32 polyols, such as from 3 to 16 carbon atoms, or from 3 to 12 carbon atoms.

By way of example, the at least one polyol, when present, may be chosen from ethylene glycol, propylene glycol, butylene glycol, hexylene glycol, glycerin, diglycerin, diethylene glycol, and dipropylene glycol, polyethylene glycols, or mixtures thereof. In some cases, the compositions include propylene glycol. In some further cases, the compositions include one or both of propylene glycol and butylene glycol. Additionally, in some cases, the composition comprises propylene glycol, and optionally at least one polyol other than propylene glycol.

Non-limiting examples of polyols that may, optionally, be included in the compositions according to the present disclosure include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and/or a mixture thereof.

In some embodiments, the at least one polyol that is optionally included is chosen from glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol, or mixtures thereof. In some cases, a composition according to the present disclosure may include at least one polyol chosen from ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, or a mixture thereof.

If present, the amount of the at least one polyol may vary but typically ranges from about 1% to about 10%, including all subranges therebetween, such as from about 0.1% to about 15%, from about 0.1% to about 12%, from about 0.1% to about 10%, from about 0.1% to about 8%, from about 0.1% to about 5%, from about 0.1% to about 4%, from about 0.1% to about 3%, from about 0.1% to about 2%, from about 0.1% to about 1.5%, from about 0.1% to about 1%, from about 0.5% to about 15%, from about 0.5% to about 12%, from about 0.5% to about 10%, from about 0.5% to about 8%, from about 0.5% to about 5%, from about 0.5% to about 4%, from about 0.5% to about 3%, from about 0.5% to about 2%, from about 0.5% to about 1.5%, from about 0.5% to about 1%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 15%, from about 1.5% to about 12%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, or from about 1.5% to about 2% by weight, relative to the total weight of the composition. For example, the at least one polyol may be present in an amount ranging from about 1% to about 9%, such as about 1.5% to about 8.5% by weight, relative to the total weight of the composition.

In some embodiments, compositions disclosed optionally comprises at least one polyol chosen from sorbitol, glycols, or mixtures thereof. When sorbitol is present, the amount of sorbitol may range from about 1% to about 3% by weight, relative to the total weight of the composition.

In some embodiments, compositions disclosed optionally comprises at least one polyol chosen from glycols. If present, the total amount of glycol(s) may range from about 1% to about 5% by weight, relative to the total weight of the composition.

### Nonionic Guar

Compositions according to the disclosure optionally comprise one or more non-ionic guar. Suitable nonionic guar may be chosen from nonionic guar gums, nonionic C1-C3 alkyl guars, nonionic C1-C3 hydroxyalkyl guarsstarches, dextrans, or mixtures thereof. In some embodiments, the compositions disclosed herein may include hydroxypropyl guar. For example, hydroxypropyl guar commercially available under the tradename JAGUAR HP 8, from the company Rhodia, may be used.

The total amount of the nonionic guar(s) in the composition disclosed herein, if present, may vary but typically ranges from about 0.1% to about 1%, including ranges and sub-ranges there between, such as, for example, from about 0.1% to about 0.8%, from about 0.1% to about 0.6%, from about 0.1% to about 0.4%, from about 0.2% to about 1%, from about 0.2% to about 0.8%, from about 0.2% to about 0.6%, from about 0.2% to about 0.4% by weight, based on the total weight of the composition.

In some embodiments, a composition according to the present disclosure includes about 0.25% by weight, relative to the total weight of the composition, of a nonionic guar such as hydroxypropyl guar.

### Additional Components

Compositions according to the disclosure may optionally comprise one or more additional components suitable for use in such compositions. Non-limiting examples of such additional components are provided below.

### pH Adjusters

Compositions disclosed herein may include one or more pH adjusters to increase or decrease the overall pH of the composition. For example, one or more acids may be included to decrease the pH of the cosmetic composition. Examples of suitable acids for decreasing the pH of the compositions may include one or more acids. The cosmetic composition may include one or more bases, such as sodium hydroxide, potassium hydroxide and the like, to increase the pH of the cosmetic composition. Additional or alternative acids and bases that are suitable for adjusting the pH of the compositions are readily known to one of ordinary skill in the art. For instance, In some embodiments, the pH adjuster is sodium hydroxide solution such as 0.1 M NaOH.

The amount of the pH adjuster in the compositions may be based on the desired pH of the final composition and/or product for improving curl definition, curl regularity, and/or curl elongation. For example, the total amount of the pH adjuster may range from about 0.0001% to about 10% by weight, including ranges and sub-ranges therebetween, based on the total weight of the cosmetic composition.

### Preservatives

One or more preservatives may be included in the compositions described herein for treating hair. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), or mixtures thereof. In some cases, the hair-treatment compositions may include one or more preservatives selected from the group consisting of sodium benzoate, benzoic acid, chlorhexidine digluconate, chlorhexidine dihydrochloride, salicylic acid, phenoxyethanol, methyl paraben, or mixtures thereof.

The total amount of the one or more preservatives, when present, may vary. In some cases, the total amount of the one or more preservatives may range from about 0.01% to about 5%, from about 0.01% to about 4%, from about 0.01% to about 2%, from about or about 0.01% to about 3% by weight, relative to the total weight of the composition.

### Auxiliary Components

Compositions according to the disclosure may optionally comprise any auxiliary component suitable for use in such compositions. Such components may include, but are not limited to, dyes/pigments for adding color to the composition, emulsifiers, moisturizing agents, fatty substances, thickeners other than those previously described, fillers, structuring agents, shine agents, antioxidants or penetrants, sequestrants, fragrances, buffers, dispersants, plant extracts, such as apricot seed powder, opacifiers, sunscreen agents, vitamins, and antistatic agents.

Optional auxiliary components may be present in an amount ranging up to about 15%, such as from about 0.001% to about 10%, from about 0.01% to about 5%, or from about 0.01% to about 1% by weight, relative to the total weight of the composition.

In various embodiments, the compositions are acidic and may have a pH of less than 7. For example, the pH of the composition may range from about 3.5 to about 6.8, from about 3.5 to about 6.5, from about 4 to about 6, including all ranges and subranges therebetween.

In some embodiments, the hair styling compositions according to the present disclosure may be free or essentially free of polyphenols including tannic acid. In further embodiments, the compositions do not include a chemical relaxing agent. In some embodiments, the hair styling compositions according to the present disclosure do not include a chemical relaxing agent. Additionally, the compositions disclosed herein may not include a cationic polymer. The compositions disclosed here may also be free or essentially free of dyes, pigments, and/or colorants.

The hair styling composition of claim 1, wherein the composition is in a gel-cream form that exhibits a texture like a gel with an opaque creamy appearance.

Compositions according to the disclosure may be in the form of a cream or lotion of water-in-oil emulsion, but are not so limited. In some embodiments, the compositions are formulated into gel-cream, which has a texture like a gel with an opaque creamy appearance. In some embodiments, the compositions are a non-aerosol type product, which may be dispensed from a bottle, tube, or spray-bottle. The compositions may be used as leave-in compositions for styling hair, particularly, straightening curly or wavy hair, without the use of heat.

### II. Methods

The compositions disclosed herein can be used for styling hair, in particular, for temporarily straightening curly or wavy hair. In addition, the compositions disclosed herein are surprisingly capable of providing a variety of additional desirable sensory properties, for example, anti-frizz, moisture, smoothness, softness, and/or shine to the hair. As such, the present disclosure also relates to methods for styling hair, particularly, temporarily straightening curly or wavy hair, while at the same time, caring for and conditioning hair, and/or imparting one or more above-described sensory benefits to the hair. Heat is not required to obtain the benefits described to the hair. As such, the methods typically do not require the use of heat.

Methods according to the disclosure typically include applying an effective amount of a composition disclosed herein to hair having waves or curls. As used herein, the term “effective amount” refers to an amount sufficient to provide a desired straightening effect to the hair, taking into account the degree of curliness, the length, the volume, and the texture of the hair, and/or an amount sufficient to provide a desired sensory benefit to the hair. In general, from about 0.5 grams to about 50 grams of product is applied to the hair, depending on the specific product formulation, hair length, hair volume, and hair style type. The composition applied to the hair may, for example, be distributed evenly by combing through with fingers or a means such as a comb or the like. After the composition is evenly distributed on the hair, the hair is allowed to air dry under an ambient condition for a period of time as needed, such as, for example, at least 15 minutes, at least 30 minutes, at least 1 hours, or up to 6 hours, until the hair is dried. In certain circumstance, instead of allowing the hair to air dry under an ambient condition, the hair may be dried with a rollerball dryer or a blow dryer on a cool setting, for example, at a temperature lower than 50° C., such as 40-45° C. After the hair is dried, the hair is combed with fingers (i.e., running fingers through the hair) or by using a comb or the like, to break the film or coating formed by the compositions on the hair for straightening the hair, and/or for a less stiff and more natural movement. Typically, no heat or chemical relaxers are applied to the hair before, during, or after the styling composition is applied to the hair.

The composition may be allowed to remain on the hair as a leave-in product for any period of time as needed, for example, a few hours or a few days, or until the next washing or rinsing of the hair. In some embodiments, the composition is allowed to remain on the hair for overnight.

In some embodiments, before applying the composition to the hair, the hair may be first cleansed with a commercially available shampoo, and/or rinsed with water. The composition may then be applied to the washed and/or rinsed hair, preferably when the hair is wet, damp, or moist. In some other embodiments, before applying the composition to the hair, if the hair is dry, the hair may optionally be moistened, damped, or wetted by water spray or using a wet towel.

Methods according to the disclosure generally do not require the use of a reducing agent, including a base, in particular a strong base (as in lye-based and no lye relaxers that employ a metal hydroxide), or heating the hair in order to achieve a temporary straightening effect. As such, in some embodiments, the method does not include using a chemical relaxing agent, a reducing agent or a base, and/or does not including heating the hair.

In various embodiments, the methods of treating hair with the compositions according to the disclosure impart the benefits described above to the hair, without a greasy feel or flaking, relative to hair not having been treated with a composition according to the disclosure. The benefits imparted to the hair may remain a desired length of time, such as a few hours, a few days, or until the hair is rinsed or washed.

The compositions described herein are not mascara compositions, i.e. are not intended to be applied to eyelashes and/or eyebrows. The compositions are likewise not makeup compositions, i.e. are not intended to be applied to skin, in particular, to skin on the face, body, or lips. Thus, treatment of, or application to, the hair is intended to be limited to hair on the head, i.e. the scalp of the head, and not eyelashes, eyebrows, body or facial hair, or the like. In such embodiments, a skilled person understands that “hair on the head” means hair that grows from the scalp of the head, and excludes eyebrows, eyelashes, facial hair, etc.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, or F may be included, one or more of a salt of A, a salt of B, a salt of C, a salt of D, a salt of E, or a salt of F may be included, or a mixture of any two of A, B, C, D, E, F, a salt of A, a salt of B, a salt of C, a salt of D, a salt of E, and a salt of F may be included. However, it is to be understood that such language also includes embodiments where G, H, I, etc., are present, as long as at least one of A, B, C, D, E, F, or a salt thereof, is also present.

The term “and/or” should be understood to include both the conjunctive and the disjunctive. For example, “A and/or B” means “A and B” as well as “A or B,” and expressly covers instances of either without reference to the other. For example, a composition that comprises “an emulsifier chosen from fatty alcohols and/or esters” means that the disclosure includes compositions comprising one or more fatty alcohols or one or more esters, as well as compositions comprising one or more fatty alcohols and one or more esters. As a further example, disclosure of “methods of styling the hair, straightening the hair, and/or reducing frizz of the hair” includes methods of styling the hair or methods of straightening the hair or methods of reducing frizz of the hair, as well as methods of styling the hair and straightening the hair, methods of styling the hair and reducing frizz of the hair, methods of styling the hair and straightening the hair and reducing frizz of the hair, etc.

As used herein, the phrases “and mixtures thereof,” “and a mixture thereof,” “and combinations thereof,” “and a combination thereof,” “or mixtures thereof,” “or a mixture thereof,” “or combinations thereof,” “or a combination thereof,” and variations thereof are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Further, unless expressly stated otherwise or when the context of the disclosure clearly dictates otherwise, the language “the at least one X may be present in an amount ranging from Y % to Z %” is to be understood to mean that the total amount of X's present in the composition ranges from Y % to Z %, inclusive of the endpoints. For example, “the at least one polysaccharide gum may be present in an amount ranging from about 0.01% to about 10%” means that the total amount of polysaccharide gums present ranges from about 0.01% to about 10%, inclusive of both 0.01% and 10%.

“Active material” as used herein with respect to the percent amount of an ingredient or raw material, refers to 100% activity of the ingredient or raw material.

All amounts given herein are relative to the amount of active material, unless otherwise indicated.

Unless otherwise indicated, all percentages herein are by weight, relative to the total weight of the composition.

All percentages, parts and ratios herein are based upon the total weight of the compositions of the present disclosure, unless otherwise indicated.

As used herein, the term “treat” (and its grammatical variations, such as “treating”) refers to the application of the compositions of the present disclosure onto the surface of the hair on the head.

As used herein, the phrase “applying a composition onto hair,” and variations thereof is intended to mean contacting the hair, with at least one of the compositions of the disclosure, in any manner. It may also mean contacting the hair in an effective amount.

As used herein, the term “styling” is intended to include “shaping” and “straightening.”

As used herein, the term “curly hair” refers to any hair including a curl, and “wavy hair” refers to any hair including a wave. The curl and/or wave may be natural or unnatural, i.e., formed by chemical treatment or physical treatment of the hair. The degree of curliness and/or waviness of the hair may vary and is not limited.

A “leave-in” composition or product refers to a hair-treatment composition that is not rinsed and/or washed away with water or acceptable solvent after the application of the composition onto the hair; instead, the composition is allowed to remain on the hair for a period of time as desired, such from 1 hour, 2 hours, 3 hours, 4 hours, up to 8 hours, overnight, or as long as needed, until next time of washing or rinsing the hair.

As used herein, the term “polyol” refers to an organic molecule comprising at least two free hydroxyl groups.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, unless expressly stated otherwise, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “salts” refers to throughout the disclosure may include salts having a counter-ion, in either ionized or un-ionized form. It is to be understood that, with regard to salts of acids described herein, it is intended to encompass the use of a salt of the acid as an ingredient added to a composition according to the disclosure, or to the salt of the acid that forms when the acid is used as an ingredient in a composition according to the disclosure (in ionized or un-ionized form).

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “temporary” should be understood to indicate that some degree of benefit or effect is imparted to the hair that remains from when the hair is treated with a composition according to the disclosure, until the composition is removed from the hair, e.g. by washing the hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition, unless otherwise noted.

### Example 1—In Vitro Hair Swatch Performance Evaluation

In vitro swatch studies were performed to evaluate the performance of the compositions according to the present.

### 1.1—Compositions

Exemplary comparative compositions C1, C2, and C3 and inventive compositions 1A and 1B were prepared according to the formulations set forth in Table 1.

TABLE 1

| | Comparative | | | Inventive | |
|---|---|---|---|---|---|
| INCI US | C1 | C2 | C3 | 1A | 1B |
| ACRYLATES COPOLYMER | 7 | 7 | 7 | 7 | 7 |
| DEHYDROXANTHAN GUM | | 1 | 1 | 1 | 1 |
| POTATO STARCH MODIFIED | | | | 0.65 | 0.65 |
| ACRYLATES/POLYTRIMETHYL | | | 1.3 | | 1.3 |
| SILOXYMETHACRYLATE | | | | | |
| COPOLYMER | | | | | |
| Preservative and Emulsifier | <0.2 | <0.2 | <0.2 | <0.2 | <0.2 |
| Solvents (water and non- | QS | QS | QS | QS | QS |
| aqueous solvent) | | | | | |

All compositions were formulated in the form of gel-cream, where compositions had textures like a gel with an opaque creamy appearance. The pH of the compositions was about 5.5-6.5 at 25° C. The compositions had Brookfield viscosities of about 24,500 cps, measured using a spindle B at 10 rpm for about 1 min under ambient conditions.

### 1.2—Hair Swatch Testing

Inventive compositions 1A and 1B and comparative compositions C1, C2, and C3 were tested against water (control) on virgin Caucasian hair swatches with curls according to the following process to evaluate the curl straightening efficacy of the compositions disclosed herein.

First, the hair swatches were washed with a commercially available shampoo in a bath ratio of about 0.4 g shampoo per gram of hair (0.4 g/g of hair). After the hair was washed, excess water was squeezed out of the swatches. Next, equal amounts of compositions 1A, 1B, and C1-C3 were respectively applied to a separate hair swatch in a bath ratio of about 0.15 g of composition per 1 g of hair, and were evenly distributed on the hair fibers by ten passes with fingers, followed by three passes with a comb to comb the hair straight. Hair swatches similarly treated with water were used as control. Photographs of these hair swatches were taken at this time point (T1). The hair swatches were then air dried at ambient condition. During drying, the hair swathes were shaken five (5) times to mimic consumer movement. Once the hair watches were dried, the swatches were evaluated for film properties (number of passes needed to break the film formed on the hair), straightness, frizziness, and tactile feel compared to the hair swatch treated with water (control) and photographs of these hair swatches were taken at this time point (T2). After the evaluation, the hair swatches were placed in a humidity chamber having 80% RH at room temperature (25° C.) for one hour. After the one-hour exposure to the high humidity condition, the hair swatches were re-evaluated for the level of straightness and frizziness, as well as tactile properties compared to the hair swatch treated using water (control). Photographs of these hair swatches were also taken at this time point (T3). The above testing was performed on hair swatches in duplicate, i.e., each composition was used to treat two separated hair swatches.

All properties were independently evaluated by ten (10) individuals. The straightness was evaluated on a scale of 1-5, with 1 the least straight and 5 the straightest. The frizziness was also evaluated on a scale of 1-5, with 1 the least frizzy and 5 the frizziest.

### 1.3—Results

The visual and tactile properties of the hair swatches evaluated by the ten individuals at the time points of after the application of the composition while the treated hair was wet (T1) and after the treated hair was dried and passed by fingers (12) were summarized in Table 2.

TABLE 2

| | | | | Tactile |
|---|---|---|---|---|
| | | | Observation | Properties of |
| | Composi- | | During | the Hair at |
| Experiment | tion | Simplex w/% AM | Application | the End |
| Ex. 1 | C 1 | Latex (7% AM) | Clean feel | 4-5 passes |
| | | | No slip | to break |
| | | | Takes | film |
| | | | some effort | Clean feel |
| | | | to distribute | |
| | | | & detangle | |
| Ex. 2 | C2 | Latex (7% AM) | Clean feel | 3-4 passes |
| | | Dehydroxanthan | Slight slip | to break |
| | | gum (1%) | Takes | film |
| | | | some effort | Slight |
| | | | to distribute | coating |
| | | | & detangle | Fibers are |
| | | | | aligned |
| EX. 3 | 1A | Latex (7%) | Slight | 3-4 passes |
| | | Dehydroxanthan | topical feel | to break |
| | | gum (1%) | Light | film |
| | | Potato Starch | coating | Straight |
| | | (0.75%) | Some drag | with some |
| | | | | fly-aways |
| Ex. 4 | C3 | Latex (7%) | Light | 3-4 passes |
| | | Dehydroxanthan | coating | to break |
| | | gum (1%) | Easier to | film |
| | | Silicone | distribute | Slight frizz |
| | | Acrylates (1.3%) | Slight slip | Some tug |
| | | | Easier to | during |
| | | | detangle | combing |
| Ex. 5 | 1B | Latex (7%) | Slight waxy | 4-5 passes |
| | | Dehydroxanthan | coating | to break |
| | | gum (1%) | Slightly | film |
| | | Potato Starch | topical | Good fiber |
| | | (0.75%) | Easy to | alignment |
| | | Silicone | detangle | |
| | | Acrylates (1.3%) | hair | |
| Control | water | | — | No film |
| | | | | Ends are |
| | | | | open |

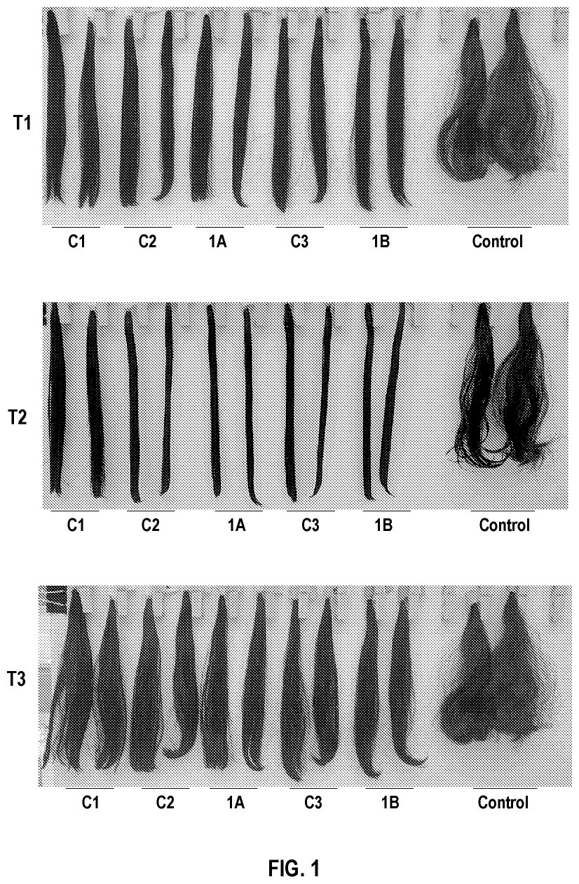

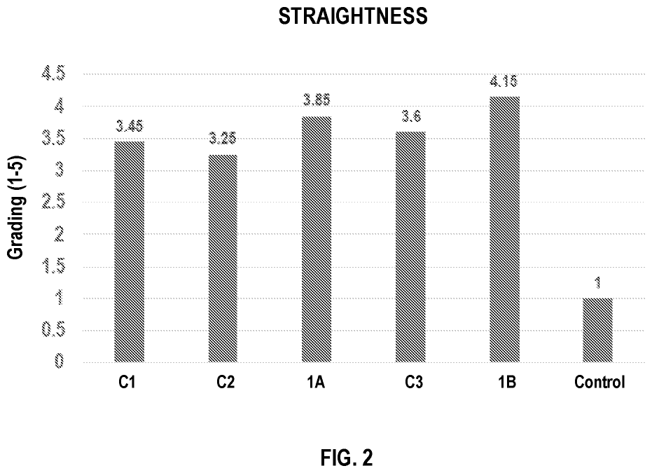

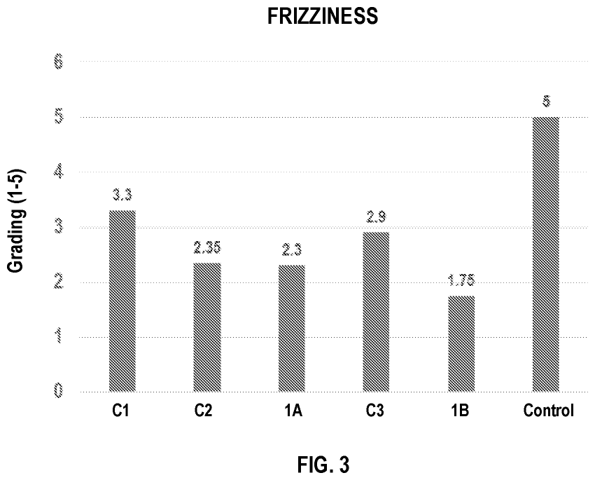

The photographs of the hair swathes at time points T1, T2, and T3 in experiments Ex.1, Ex.2, Ex.3, Ex.4, and Ex.5, corresponding to the hair respectively treated with compositions C1, C2, 1A, C3, and 1B, against the hair swatches treated with water (control), was shown in the photographs in FIG. 1. The graded levels of straightness and frizziness of the hair at T3 are illustrated in FIG. 2 and FIG. 3, respectively.

As can be seen in FIGS. 1, 2, and 3, the hair swatches treated with compositions C1, C2, C3, 1A, and 1B all showed noticeable straightening and frizz control properties over control. The properties lasted even after the hair was exposed to high humidity condition (80% RH) for an hour. The hair swatches treated with composition 1B, which included acrylates copolymer, dehydroxanthan gum, potato starch, and silicone acrylates, were the straightest with the least amount of frizz. However, 4-5 passes were needed to break the film on the hair treated with composition 1B, which was more than the number of passes required to break the films formed on the hair by composition 1A, C2, and C3, as shown in Table 2. As such, the hair treated with composition 1B was stiffer than the hair treated with compositions 1A, C2, and C3. In contrast, the hair treated with composition 1A, which included acrylates copolymer, dehydroxanthan gum, and potato starch but not silicone acrylates, had a film that was easier to break (3-4 passes to break), good fiber alignment, and more natural look and feel, as preferred by the consumers, compared to the hair treated with composition 1B. In addition, the hair swatches treated with composition 1A had better straightness and was less frizzy compared to the hair treated with compositions C1, C2, and C3, which do include a starch.

### Example 2—In Vivo Evaluation on Consumer Hair

In this example, the performance of compositions according to the present disclosure having different levels of acrylates copolymers was tested and evaluated in vivo on a consumer's hair.

### 2.1—Compositions

Compositions 2A and 2B according to the present disclosure with different amounts of acrylates copolymer as set forth in Table 3 were prepared.

TABLE 3

| | Inv. | Inv. |
|---|---|---|
| INCI US | 2A | 2B |
| ACRYLATES COPOLYMER (Latex) | 10 | 7 |
| DEHYDROXANTHAN GUM | 0.75 | 0.75 |
| POTATO STARCH MODIFIED | 0.65 | 0.65 |
| DIMETHICONE | 3.5 | 3.5 |
| AMODIMETHICONE | 4 | 4 |
| BUTYLENE GLYCOL | 4.0 | 4.0 |
| CAPRYLYL GLYCOL | 0.3 | 0.3 |
| HYDROXYPROPYL GUAR | 0.25 | 0.25 |
| HYDROGENATED STARCH HYDROLYSATE | 1.65 | 1.65 |
| Preservatives, pH Adjuster, Stabilizers, Emulsifiers, etc. | <2 | <2 |
| Solvents(Water and Non-Aqueous Solvent) | QS | QS |

### Preparation Process:

Side Kettle 1: Add 8% of water into suitable beaker. Sprinkle potato starch and start heating to 80° C. to 85° C.
Side Kettle 2: Add amodimethicone (and) trideceth-6 (and) cetrimonium chloride and dimethicone in a suitable beaker and mix until uniform.
Main Kettle:
   1) Add 49% of water.
   2) Sprinkle hydroxypropyl guar and dehydroxanthan gum slowly with aggressive agitation and mix until uniform.
   3) Once uniformed, add butylene glycol, hydrogenated starch hydrolysate, phenoxyethanol, caprylyl glycol, ethylhexylglycerin, and the content of side kettle 2, and mix until uniform.
   4) Transfer the content of side kettle 1 to the main kettle, using 3% of water during transferring.
   5) Filter acrylates copolymer through 50 micron filter.
   6) Add filtered acrylates copolymer and remaining water into the main kettle.
   7) Mix until uniform.
   8) Take specifications.

Compositions 2A and 2B were in the form of a gel-cream and had viscosities of about 24,500 cps, when measured using a Brookfield rheometer with a spindle B at 10 rpm for 1 minute under the ambient condition. The pH of these compositions was about 5.5 to about 6.5 at 25° C.

### 2.2—Hair Swatch Testing

Compositions 2A and 2B were tested in vivo on a consumer having curly hair with average hair density, diameter, and porosity.

Before starting the treatment, photographs were taken to show the initial look of the hair on the left side and right side of the consumer' head. After the initial photographs were taken, the consumer's hair was washed with a commercially available shampoo and then conditioned using a commercially available lightweight conditioner. The washed hair was diffused to approximately 80% dry. After then, 15 gram of each of compositions 2A and 2B was respectively applied in sections on the consumer's hair via half head application, where composition 2A was applied on the left side from back view and composition 2B was applied on the right sight from back view. After the applications, the hair on each side of the head was separately combed through to straighten, followed with air drying. After air-drying for approximately 4 hrs, the hair was evaluated for straightness and tactile feel, and photographs were taken to show final look of the hair on the left side and right side of the consumer' head at the end of the treatment (T1).

### 2.3—Results

It was observed that during application, both compositions were easy to apply and easy to distribute with a good balance of slip and grip to help create a straight style during combing, with a slight preference by the consumer for composition 2B, which has less acrylates copolymer (latex) than composition 2A. In addition, it was observed that after the treated hair was dried and combed through, the straight style was maintained. When the treated hair was dried, the hair also exhibited improved root volume and hair movement, as well as other properties such as less frizzy, clean feel, lightweight, and smoothness.

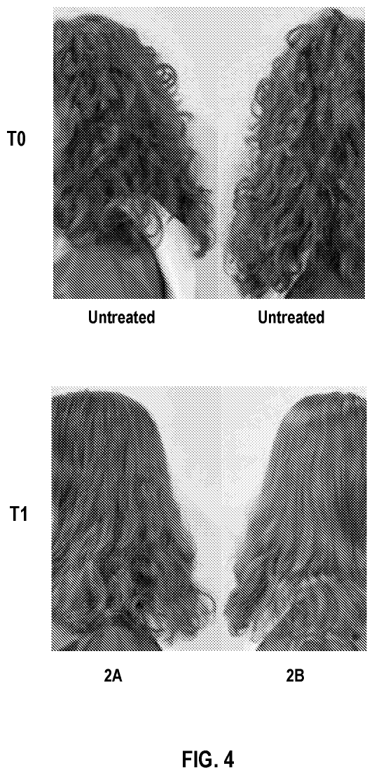

FIG. 4 is a set of the photographs showing the appearance of the consumer's initial hair before the hair was treated (TO) and the final look after the hair was treated (T1). The hair on the left side (back view) was treated with composition 2A, and the hair on the right side (back view) was treated with composition 2B. As shown in FIG. 4, compositions 2A and 2B both provided noticeable heatless (no heat was added to the hair) straightening and frizz control benefits to the hair. The hair treated with composition 2B was slightly less frizzy and had better discipline, compared to the hair treated with composition 2A.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1. A hair styling composition comprising:
(a) at least one latex film former chosen from acrylate-based polymers, polyurethane-based polymers, derivatives thereof, or combinations thereof;
(b) at least one polysaccharide gum;
(c) potato starch and/or a derivative thereof; and
(d) water;
wherein the pH of the hair styling composition is less than 7.

2. The hair styling composition of claim 1, wherein the total amount of the latex type film former(s) chosen from acrylate-based polymers, polyurethane, derivatives thereof, or combinations thereof ranges from about 0.5% to about 15% by weight, relative to the total weight of the composition.

3. The hair styling composition of claim 2, wherein the acrylate-based polymers or derivatives thereof are chosen from acrylates copolymer of two or more monomers of (meth)acrylic acid or one of their simple ester, styrene/acrylates copolymer, acrylates/ethylhexyl acrylate copolymer, or mixtures thereof.

4. The hair styling composition of claim 1, wherein the at least one polysaccharide gum is chosen from sclerotium gum, gum arabic, tragacanth gum, karaya gum, guar gum, gellan gum, tara gum, locust bean gum, tamarind gum, xanthan gum, dehydroxanthan gum, carrageenan gum, Seneca gum, gellan gum, or mixtures thereof.

5. The hair styling composition of claim 1, wherein the total amount of polysaccharide gum(s) ranges from about 0.1% to about 2% by weight, relative to the total weight of the hair styling composition.

6. The hair styling composition of claim 1, wherein the total amount of potato starch and/or derivatives thereof ranges from about 0.2% to about 5% by weight, relative to the total weight of the hair styling composition.

7. The hair styling composition of claim 1, wherein the amount of water is at least about 70% by weight, relative to the total weight of the composition.

8. The hair styling composition of claim 1, further comprising (e) at least one silicone acrylate, wherein the amount of silicone acrylate ranges from about 0.5% to about 20% by weight, relative to the total weight of the hair styling composition.

9. The hair styling composition of claim 1, wherein the composition further comprises at least one silicone oil, wherein the total amount of silicone oil(s) ranges from about 2% to about 10% by weight, relative to the total weight of the composition.

10. The hair styling composition of claim 9, wherein the at least one silicone oil is chosen from methicone, amodimethicones, dimethicones, polydiethylsiloxanes, dimethiconols, or mixtures thereof.

11. The hair styling composition of claim 1, further comprising at least one polyol; wherein the total amount of polyol(s) ranges from about 1% to about 10% by weight, relative to the total weight of the composition.

12. The hair styling composition of claim 11, wherein the at least one polyol is chosen from sorbitol, glycols, or mixtures thereof; wherein when sorbitol is present, the amount of sorbitol ranges from about 1% to about 3% by weight, relative to the total weight of the composition; wherein when at least one glycol is present, the total weight of glycol(s) ranges from about 1% to about 5% by weight, relative to the total weight of the composition.

13. The hair styling composition of claim 1, wherein the composition further comprises at least one non-ionic guar, wherein the total amount of non-ionic guar(s) ranges from about 0.1% to about 1% by weight, relative to the total weight of the composition.

14. The hair styling composition of claim 1, wherein the composition is free or substantially free of a polyphenol.

15. The hair styling composition of claim 1, wherein the composition is free or substantially free of chemical relaxing agents.

16. The hair styling composition of claim 1, wherein the composition is in a gel-cream form that exhibits a texture like a gel with an opaque creamy appearance.

17. A hair styling composition comprising:
(a) at least one latex type film former chosen from acrylate-based polymers, polyurethane-based polymers, derivatives thereof, or combinations thereof, present in an amount ranging from about 1% to about 15% by weight;
(b) at least one polysaccharide gum, present in an amount ranging from about 0.1% to about 2% by weight;
(c) potato starch or a derivative thereof, present in an amount ranging from about 0.2% to about 5% by weight;
(d) optionally, a silicone acrylate, present in an amount ranging from about 0.5% to about 20% by weight;
(e) optionally, at least one polyol, present in an amount ranging from about 1% to about 10% by weight;
(f) optionally, at least one non-ionic guar, present in an amount ranging from about 0.1% to about 1% by weight;
(g) optionally, at least one at least one silicone oil, present in an amount ranging from about 2% to about 10% by weight, relative to the total weight of the composition; and
(h) water, present in an amount of at least 70% by weight;
wherein the pH of the hair styling composition is less than 7; and
wherein all amounts are relative to the total weight of the composition.

18. A method for temporarily straightening hair comprising:
applying to the hair a hair styling composition comprising:
(a) at least one latex type film former chosen from acrylate-based polymers, polyurethane-based polymers, derivatives thereof, or combinations thereof;
(b) at least one polysaccharide gum;
(c) potato starch or a derivative thereof;
(d) water;
(e) optionally, at least one silicone acrylate copolymer;
(f) optionally, at least one silicone oil;
(g) optionally, at least one polyol; and
(h) optionally, at least one non-ionic guar;
wherein the pH of the hair styling composition is less than 7; and
after the hair styling composition is applied, straightening the hair with fingers or a combing tool.

19. The method of claim 18, wherein the hair styling composition is left on the hair for a period of time until the hair is washed.

20. The method of claim 18, wherein no heat or chemical relaxers are applied to the hair before, during, or after the hair styling composition is applied to the hair.
